(19) United States
(12) Patent Application Publication
(10) Pub. No.: US 2014/0163047 A1
(43) Pub. Date: Jun. 12, 2014

(54) COMPOSITION OF A CERAMIC MATRIX WITH A CONTROLLED RELEASE DRUG, A TABLET OBTAINED FROM THE COMPOSITION AND METHODS FOR OBTAINING THE COMPOSITION AND THE TABLET

(71) Applicant: Instituto Presbiteriano Mackenzie, Sao Paulo (BR)

(72) Inventors: Antonio Hortencio Munhoz, JR., Santo Andre (BR); Richard Wagner Novickis, Sao Paulo (BR); Leila Figueiredo De Miranda, Sao Paulo (BR); Sonia Braunstein Faldini, Sao Paulo (BR); Mauro Cesar Terence, Sao Paulo (BR); Roberto Rodrigues Ribeiro, Sao Paulo (BR)

(73) Assignee: Instituto Presbiteriano Mackenzie, Sao Paulo (BR)

(21) Appl. No.: 14/096,770

(22) Filed: Dec. 4, 2013

Related U.S. Application Data
(63) Continuation-in-part of application No. 12/928,546, filed on Dec. 13, 2010, now abandoned.

(30) Foreign Application Priority Data
Dec. 21, 2009 (BR) PI0906820-1

(51) Int. Cl. A61K 47/02; A61K 31/52

(52) U.S. Cl. CPC A61K 47/02; A61K 31/52
USPC 514/263.4

(57) ABSTRACT

The present invention refers to a composition of a ceramic matrix defined by pseudoboehmite nanoparticles and constructed for carrying and releasing medications in a controlled manner, in the treatment of human beings and animals presenting an organic deficiency which requires the application of said medications. The present invention further refers to the method for preparing said composition, in the form of a ceramic matrix, and also to the method of incorporating the drug acyclovir to said ceramic matrix, forming a tablet.

### FIELD OF THE INVENTION

The present invention refers to a ceramic nanosystem composition for releasing acyclovir and similar drugs in a controlled manner, in the treatment of human beings and animals presenting an organic deficiency which requires the application of said medication. The present invention further refers to the method for preparing said nanosystem composition, in the form of a ceramic matrix incorporating the active compound or drug, and also to the method of producing a tablet from said composition.

### BACKGROUND OF THE INVENTION

Many of the drugs employed in the treatment of several diseases are indiscriminately distributed in several organs and tissues after administration thereof, which can cause inactivation of said drugs or undesirable effects not related to the pathological process. Besides, as a consequence of this wide distribution, for achieving the therapeutic concentration required in a certain organ or part of the organism, it is necessary to administer large amounts of the therapeutic agent.

Aiming at overcoming these drawbacks, there have been developed new drug-carrier systems to rationalize the medication therapy, leading to the reduction of the dose and of the undesired side effects, as well as stimulating the patient to adhere to the treatment.

The drug vectorization, based on Paul Ehrlich's the theory about the ability of tiny particles in carrying active molecules to the specific action sites, has been considered one of the major biopharmaceutical research lines of the last decades, taking part in a wide-range area, denominated nanotechnology, which quickly emerged in Brazil and in the world. It is a consensus that the use of nanostructured colloidal systems, such as the liposomes, nanoemulsions and polymeric nanoparticles, is an alternative which aims to alter the biodistribution of drugs after administration thereof by different routes. The vector-oriented release system delivers, selectively, the drug to its action site, in order to offer the maximum therapeutic activity, prevent the degradation or inactivation during the transit until the target site, and protect the body from adverse reactions due to the inappropriate distribution (BANKER and RHODES, 1996).

Many pathologies present potential for treatment through drug vectorization such as, for example, parasite infections in cells of the endothelial reticulum system, diseases that affect the central nervous system, tumors, and the like. Specifically for cancer, the increase of the vascular permeability of the tumor tissue enables extravasation of the drug carriers presenting between 10 and 700 nanometers of diameter. This increase of the capillary permeability results from the poor formation of the neo-vasculature of the tumor tissues, which present gaps between the endothelial cells.

Thus, the application of the vectorized transport systems has potential to improve, for example, the chemotherapy of neoplasias. The effective use of said systems would, not only reduce the chemotherapeutic agent dose for a given degree of therapeutic answer, but also improve the opportunities for some cells which are typically resistant to certain drugs. Moreover, the chemotherapy application via these systems could reduce the complexity of the surgical manipulation, minimizing the severity of the cancer extension and/or reducing the residual volume. Alternatively, the use of these systems, after the tumor has been reduced through surgery and/or radiation therapy, allows enhancing the probability of effectively eradicating the residual cancerous cells (GUPTA, 1990).

Another application of said technology might be noted in the international publication WO 2008/069561, which refers to a metal oxide hollow nanocapsule capable of carrying a drug adsorbed in its structure.

This prior art solution requires the provision of a nanocapsule surrounding the drug to be released in a predetermined organic medium, through the wall of the shell defined by said hollow nanocapsule. In this case, the drug is not incorporated in the metal oxide matrix itself, but enclosed in its interior.

The construction of the hollow nanocapsule requires specific and complex procedures, which demand sophisticated equipment and lead to high production costs.

Besides the above-cited drawback, the solution described in the international patent application mentioned above also requires that the metal oxide nanocapsule be surrounded by a silica coating to keep the drug contained in the interior of the nanocapsule, until the latter reaches the region of the organism able to remove the silica coating and allow the drug to be controllably and progressively released through the surrounding wall of the nanocapsule containing the drug.

The provision of the silica coating is fundamental to prevent undesired aggregations to the nanocapsule wall, which aggregations, without the provision of the coating, require the use of aqueous dispersions containing electrostatic stabilizers, surfactants, polymers, such as steric stabilizers and polymer modelers.

These aspects make it even more costly and complex the use of metal oxide nanocapsules encapsulating the drugs to be released.

For example, the drug acyclovir which is used against types I and II of simple herpes and zoster virus is poorly soluble in water, insoluble in alcohol and only slight soluble in acid or diluted alkaline solutions.

In relation to the dissociation constant, pKa, presents two pH values: 2,3 and 9,2, that is in said two pH values we have 50% of said molecules in the ionic form and 50% in the molecular.molecular form.

The solubilization of the acyclovir is difficult, which results in a low absorption by the gastro-intestinal tract of a human or an animal.

When orally administrated the acyclovir is partially absorbed in the gastro-intestinal tract. In fact, only 20% of the administered dose are absorbed by the organism and the maximum plasmatic concentration are reached from 1 to 2 hours. Normally, it is required that the acyclovir be administered twice a day.

Considering the low solubilization of the acyclovir and the consequent reduced absorption thereof by the organism, the doses administered to a patient has to contain necessarily an excess of the drug in order to allow that the organism receive the minimum adequate amount of the drug. The non-proportional amount of the drug not absorbed by the organism is, despite its elevated cost delivered thereof without producing any positive effect.

From the deficiency of solubilization of the acyclovir, it is naturally desirable to provide a reliable, efficient and economically feasible vehicle to allow the administration to a human or an animal the required amount of the acyclovir released in a controlled manner.

### SUMMARY OF THE INVENTION

As a function of the drawbacks pointed above, the present invention has the object of providing a composition of a ceramic matrix with a controlled release drug, a tablet obtained from the composition and methods for obtaining the composition and the tablet, said ceramic matrix carrying a low-soluble drug and allowing the latter to be controllably released in the organism in which the composition is administered, allowing that the amount of the drug administered to the organism is that one required and effectively absorbed by the latter.

According to a first aspect of the invention, the ceramic matrix of the composition is formed by pseudoboehmite nanoparticles, presenting a pharmaceutically acceptable degree of purity, a specific area of 250-300 mg²/g and defining 50% to 60% of the total composition weight, the drug comprising acyclovir to be controllably released in a human or animal organism and defining at least a portion of the remaining weight of the composition.

According to a second aspect of the invention, the ceramic matrix of the tablet is formed by pseudoboehmite nanoparticles, presenting a pharmaceutically acceptable degree of purity, a specific area of 250-300 mg²/g and defining 50% to 60% of the total tablet weight, the drug comprising acyclovir to be controllably released in a human or animal organism and defining at least a portion of a remaining weight of the tablet.

According to a third aspect of the invention, the composition is obtained by a method comprising, in a first phase, the production of the ceramic matrix through the steps of:

mixing an aqueous aluminium nitrate or an aqueous aluminium chloride solution (14% m) with a poly(vinyl alcohol) solution (8% m in water), forming a precursor solution;

dripping the precursor solution in an ammonium hydroxide solution (28% m), forming a gel;

ageing the gel, filtering and drying it by about 70° C. for approximately 24 hours to obtain a ceramic matrix of pseudoboehmite presenting a specific area of 250-300 m²/gram; and, in a second phase, the step of

mixing the ceramic matrix of pseudoboehmite, in an amount from 50% to 60% of the total composition weight, with a controlled release drug comprising acyclovir to be controllably released in a human or animal organism and defining at least a portion of the remaining weight of the composition.

The invention also refers to a method for producing the tablet, comprising, in a first phase, the production of the ceramic matrix, through the steps of:

mixing an aqueous aluminium nitrate solution or aqueous aluminium chloride solution (14% m) with a poly(vinyl alcohol) solution (8% m in water), forming a precursor solution;

dripping the precursor solution in an ammonium hydroxide solution (28% m); forming a gel;

ageing the gel, filtering and drying it at about 70° C. for approximately 24 hours to obtain a ceramic matrix of pseudoboehmite presenting a specific area of 250-300 m²/gram; and in a second phase, the steps of

mixing the ceramic matrix of pseudoboehmite, in an amount from 50% to 60% of the total tablet weight, with a controlled release drug comprising acyclovir to be controllably released in a human or animal organism and defining at least part of the remaining weight of the tablet; and

submitting the mixture ceramic matrix/acyclovir to a conformation under a pressure sufficient to form the tablet.

The use of a ceramic matrix of pseudoboehmite nanoparticles for carrying the drug acyclovir allows the pseudoboehmite, with its high specific area, to increase the solubility degree of the acyclovir in the gastro-intestinal tract, allowing the use of doses for example in the form of tablets, containing, in a more precise manner, the amount of the acyclovir simultaneously required and absorbed by the organism in which it is orally administered.

### BRIEF DESCRIPTION OF THE FIGURES

FIG. 1a is a photograph taken by scanning electron microscope of a liver of an animal from a control group;

FIG. 1b is another photograph of the liver of the animal from the control group;

FIG. 1c is a photograph of a liver of an animal from a first experimental group;

FIG. 1d is another photograph of the liver of the animal from the first experimental group;

FIG. 1e is a photograph of a liver of an animal from a second experimental group;

FIG. 1f is another photograph of the liver of the animal from the second experimental group;

FIG. 1g is a photograph of a liver of an animal from a third experimental group; and

FIG. 1h is another photograph of the liver of the animal from the third experimental group.

### DETAILED DESCRIPTION OF THE INVENTION

As described above, the invention includes the provision of a composition comprising a ceramic matrix and a controlled release drug, the ceramic matrix having a structure formed by pseudoboehmite nanoparticles, presenting a specific area of 250-300 m²/gram.

This large specific area of the ceramic matrix of pseudoboehmite nanoparticles allows said matrix to incorporate, in small material volumes, usually defined in tablets to be ingested by the human being or animal, a large quantity of one or more drugs, generally incorporating a pharmaceutically acceptable filler and also at least one flow adjusting element and a lubricant agent which facilitates the final compression of the composition defined by the ceramic matrix and the drug, for forming a tablet.

Thus, the invention allows obtaining a composition and also a tablet comprising a ceramic matrix formed by pseudoboehmite nanoparticles, presenting a specific area of 250-300 mg²/g and defining 50% to 60% of the total composition or tablet weight; and a drug comprising acyclovir to be controllably released in a human or animal organism and defining at least a portion of the remaining weight of the composition or the tablet.

In the case of the oral administration of the tablet the controlled and progressive release of the drug from the pseudoboehmite matrix is obtained through the structural collapse of the tablet inside the organism in which it is administered.

In the preferred way of carrying out the invention, the composition comprises not only the acyclovir, but also a pharmaceutically acceptable filler, a flow adjusting element and a lubricant agent used in the compression phase of the formation of the tablet.

The drug used in the composition is defined by the acyclovir compounds and usually provided in a dose of 100 mg.

The pharmaceutically acceptable filler may be defined by starch, which is present in the composition in an amount ranging from 20% to 30%.

The flow adjusting element is generally defined by silicon dioxide, which is present in an amount which ranges from 1.5% to 2% in relation to the total weight of the pharmaceutical composition.

The lubricant agent may be defined by magnesium stearate, which is present in the pharmaceutical composition in an amount ranging from 1.5% to 2%.

For obtaining the composition of a ceramic matrix with a controlled release drug the invention provides a method which comprises, in a first phase, the production of a ceramic matrix of pseudoboehmite nanoparticles through the steps of:

mixing an aqueous aluminium nitrate or an aqueous aluminium chloride solution (14% m) with a poly(vinyl alcohol) solution (8% m in water), forming a precursor solution;

dripping the precursor solution in an ammonium hydroxide solution (28% m), forming a gel;

ageing the gel, filtering and drying it by about 70° C. for approximately 24 hours to obtain a ceramic matrix of pseudoboehmite presenting a specific area of 250-300 m²/gram; and, in a second phase, the step of

mixing the ceramic matrix of pseudoboehmite, in an amount from 50% to 60% of the total composition weight, with a controlled release drug comprising acyclovir to be controllably released in a human or animal organism and defining at least a portion of the remaining weight of the composition.

For obtaining a tablet comprising the composition as defined above, it is applied a method which comprises, in a first phase, the production of a ceramic matrix of pseudoboehmite nanoparticles, through the steps of:

mixing an aqueous aluminium nitrate solution or aqueous aluminium chloride solution (14% m) with a poly(vinyl alcohol) solution (8% m in water), forming a precursor solution;

dripping the precursor solution in an ammonium hydroxide solution (28% m), forming a gel;

ageing the gel, filtering and drying it at about 70° C. for approximately 24 hours to obtain a ceramic matrix of pseudoboehmite presenting a specific area of 250-300 m²/gram; and in a second phase, the steps of

mixing the ceramic matrix of pseudoboehmite, in an amount from 50% to 60% of the total tablet weight, with a controlled release drug comprising acyclovir to be controllably released in a human or animal organism and defining at least part of the remaining weight of the tablet; and

submitting the mixture ceramic matrix/acyclovir to a conformation under a pressure sufficient to form the tablet.

Preparation of the Tablets:

The procedures related to the production of tablets using the acyclovir drug will be commented below.

There were produced lots of acyclovir tablets adsorbed with the pseudoboehmite, for conducting in-process dissolution and control tests. Another lot was made using a physical mixture of the drug and of the ceramic material, in predefined proportions.

The preparation of the tablets was carried out by direct compression, through the rotating press (brand Lemaq-model Mini Express L.N.S.), according to a formulation as exemplified below:

Acyclovir=quantity sufficient to form a dose of 100 mg;

Starch—30% of the total tablet formulation;

Colloidal silicon dioxide (Aerosil 200)=2% of the total tablet formulation; and

Magnesium stearate=2% of the total tablet formulation;

Pseudoboehmite=50 to 60% of the total tablet formulation.

Procedure:

Mixing the formulation components (ceramic matrix/acyclovir/starch/colloidal silicon dioxide), except the magnesium stearate, in a V-shaped mixer (brand Lemaq—model M “V”), for at least 15 minutes.

Adding the magnesium stearate and mixing for at least 5 minutes. Transferring the mixture to the press-forming machine, with the aid of a scoop. Pressing the mixture in a 10 mm punch. Proceeding to the in-process control tests (average mass, friability and hardness).

According to the invention, and considering the pseudoboehmite synthesis study previously carried out at the Material Characterization Laboratory of the Universidade Presbiteriana Mackenzie (Mackenzie Presbyterian University) (CARRIO, 2007; MUNHOZ JR, 2006), pseudoboehmites were synthesized from two precursors AlCl3 and Al(NO3)3.9H2O. The samples obtained were structurally analyzed and used as a support for the production of nanoparticulate systems containing bioactive molecules.

The evaluation of the systems produced as drug carriers was conducted through interaction tests, by using the techniques of UV-VIS spectrometry, scanning electron microscopy, X-ray diffraction and spectrophotometry in the infrared region.

Preparation of the Pseudoboehmites

As already previously cited, the used reagents are aqueous aluminium nitrate solution (Al(NO3)3.9H2O), aqueous aluminium chloride solution, aqueous ammonium hydroxide solution (NH4OH) (14% m and 28% m) and aqueous poly(vinyl alcohol) solution (8% m in water).

The poly(vinyl alcohol) solution was used to increase the viscosity of the aluminium nitrate or aluminium chloride solution.

The aluminium nitrate or aluminium chloride solution is mixed to the poly(vinyl alcohol) solution, forming a precursor solution which is then dripped in the ammonium hydroxide solution, forming a gel. After ageing the gel, it is filtered in a Buchner funnel and dried at 70° C. for 24 hours.

Incorporation of the Acyclovir to the Pseudoboehmite

The incorporation of the acyclovir to the ceramic matrix to form the composition of the invention is conducted through the solubilization of the active principle in an appropriate solvent, followed by addition of the pseudoboehmite. The mixture is maintained under constant agitation, at a determined temperature, during a given period of time.

All the experimental conditions are optimized with the purpose of searching for a greater interaction between the acyclovir molecule and the ceramic material, in a shorter time and at a lower temperature for the test. After the incorporation of the active principle, the mixture is centrifuged and the supernatant analyzed by UV-VIS spectrophotometry, for determining the quantity of acyclovir molecules which interacted with the ceramic material.

The dispersion is filtered and the resulting material is washed and dried to be used in posterior analytic procedures.

Interaction Test

Scanning electron microscopy, UV-VIS spectrophotometry, X-ray diffraction and infrared spectroscopy are the techniques used to confirm the interaction of the acyclovir molecules with the pseudoboehmite ceramic matrix.

Scanning Electron Microscopy: Direct Determination of the Interaction Process Between Acyclovir/Pseudoboehmite.

The scanning electron microscopy (SEM) is a technique which allows analyzing, visually, the spatial distribution of the particulate matters and, therefore, aids in analyzing the acyclovir/pseudoboehmite interaction process, contributing to the analysis of the uniformity of its distribution and to the homogeneity of the inorganic crystals of the ceramic material. The SEM provides information about the diameter of the particulate materials and about the reproducibility of the synthesis conditions, thus allowing adjusting and improving these procedures.

UV-VIS Spectrometry: Determination of the Adsorption of the Acyclovir to the Pseudoboehmite.

The quantification of the active component to be adsorbed by the ceramic material may be evaluated through the ultraviolet UV-VIS spectrophotometry, via calibration curve of each of the substances in the appropriate solvent for the adsorption test and in the more adequate wave length for each substance.

The optimization of the test conditions can be obtained by analyzing the conditions which most favor the adsorption. The parameters to be optimized are: total test time, temperature and the relation of concentration between the active principle of the acyclovir and the pseudoboehmite.

Through the analysis by UV-VIS, it can be determined the amount of active component which was not adsorbed by the matrix and, by comparing these data with the previously obtained calibration curve, one can indirectly find the concentration of bioactive molecules which were adsorbed by the ceramic matrix.

Thus, it is possible to evaluate, for example, the pseudoboehmite/acyclovir interaction and to know its adsorption yield.

X-Ray Diffraction: Determination of the Interaction Process Between Acyclovir and Pseudoboehmite

It should be emphasized that an X-ray diffraction equipment provides qualitative and quantitative information about the obtained structure and about the acyclovir/pseudoboehmite nanointeractions.

Absorption Spectroscopy in the Infrared Region: Determination of the Interaction Process Between Acyclovir and Pseudoboehmite

The analysis by spectrophotometry in the infrared region can provide information about the adsorption mechanism, comparing the infrared spectra of the adsorbed acyclovir and of the pure acyclovir. It is possible to verify the absorption displacement of some groups of adsorbed acyclovir by influence of the ceramic material (WHITE & HEM, 1983).

The production of pseudoboehmite through the sol-gel process, from high-purity reagents, makes possible to obtain high-purity grade pseudoboehmite presenting a high specific area and being totally devoid of contaminants, making it, therefore, adequate for applications in controlled release of drugs. In view of the possible use of pseudoboehmite as excipient for controlled release of drugs, tests were conducted in order to determine its acute toxicity (50 mg/kg, 300 mg/kg, and 2000 mg/kg) and sub-acute toxicity (1000 mg/kg) in Wistar rats. The tests were performed according to Organization for Economic Cooperation and development OECD 423 guide. The methodology consisted of toxicity by evaluating and analyzing of the biochemical and histopathological parameters which resulted from administration thereof. Finally, pseudoboehmite was tested in vivo as a possible controlled releaser of the acyclovir drug. In both tests' the administration did not determine mortality in the groups. Furthermore, no changes were observed in the tissue integrity during the histopathological evaluation of the animal livers.

Macroscopic and Histopathological Evaluation—Acute Oral Toxicity.

No abnormal changes were observed during macroscopic tests of the organs of the animals.

In the slices of the liver of the Wistar rats, it was evaluated the presence of hepatic necrosis, proliferation of biliary ducts, proliferation of fibrous conjunctive tissue, and blood extravasation (Cunha, et. al., 2009).

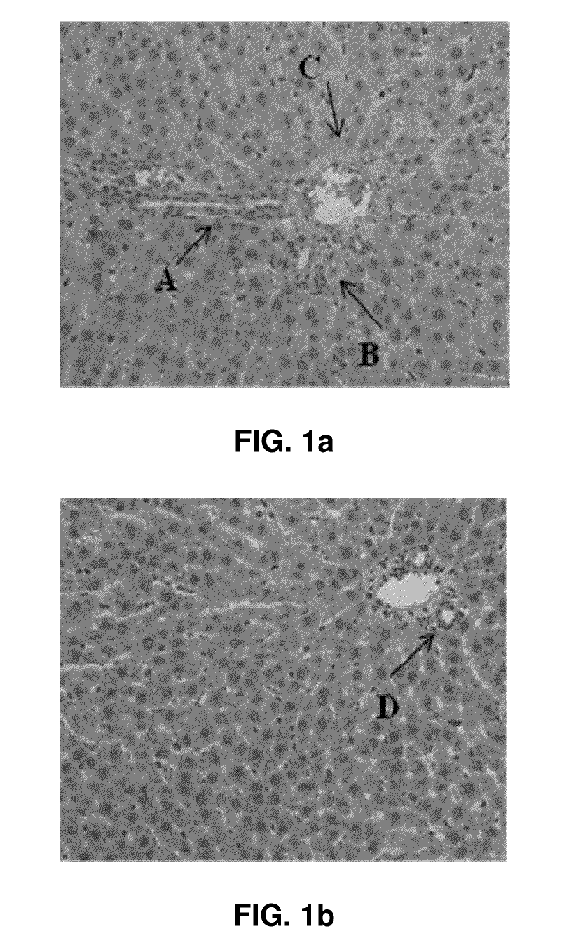

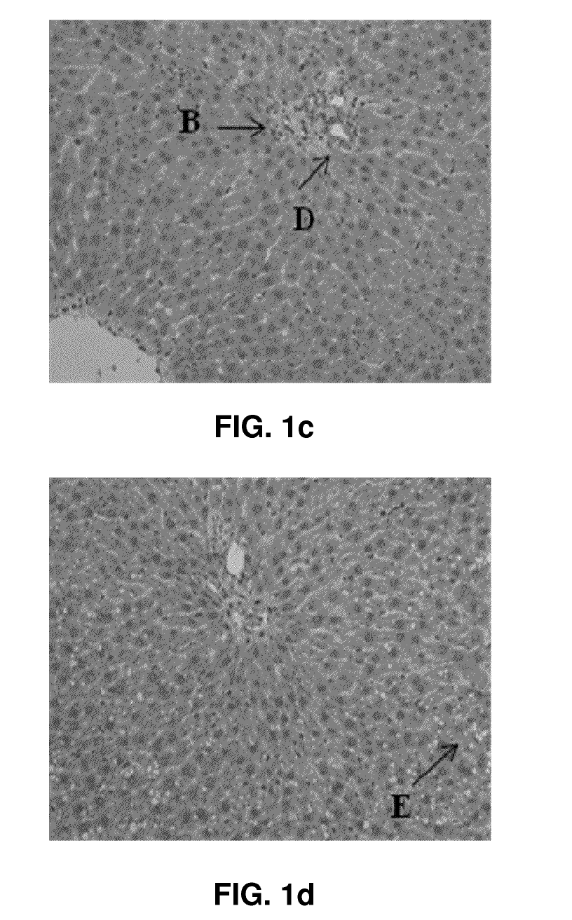

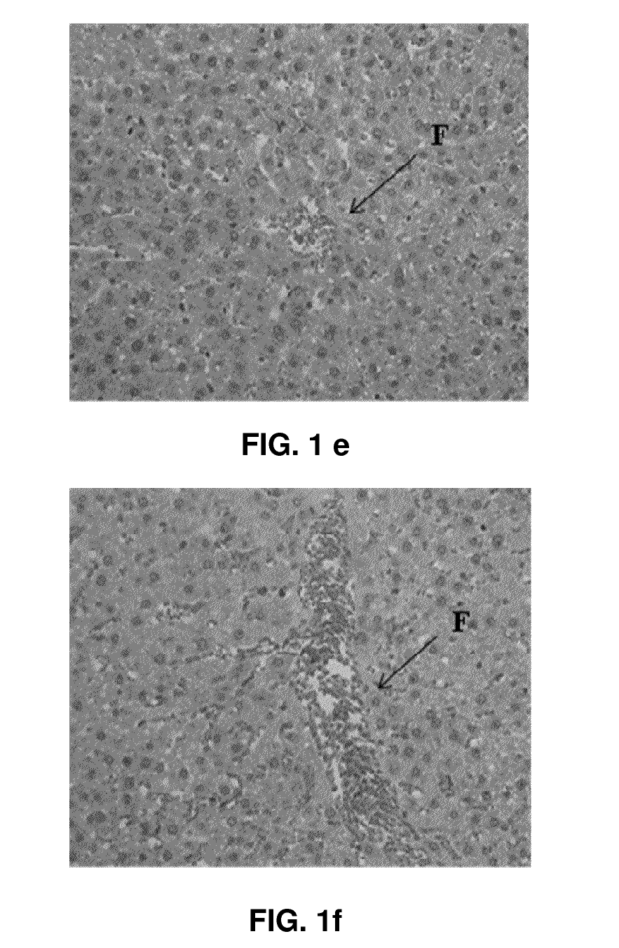

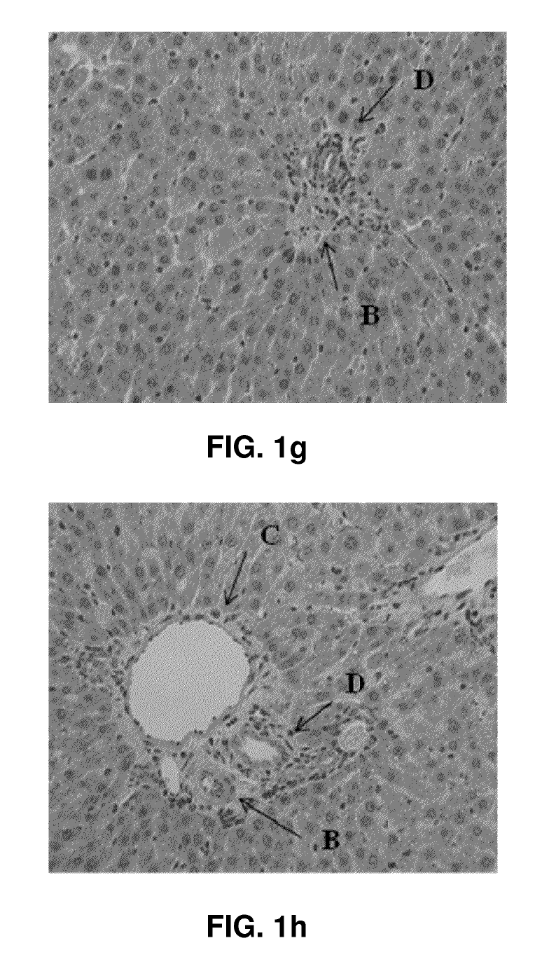

During the histopathological evaluation of the livers of the animals, there were not observed any changes in the tissue integrity or apparent injuries in the different experimental groups. However, in one of the rodents from group 2 (single dose of 300 mg/kg), a sinusoidal congestion was detected (see attached FIG. 1f). The occurrence of this congestion in one single animal of the group excludes the administration of pseudoboehmite as being its cause.

FIGS. 1a to 1h of the drawings refer to the histopathological analysis of the acute oral toxicity of the pseudoboehmite in the control and experimental groups (increase: 200×).

FIG. 1a shows a liver of the animal from the control group, evidencing tissue integrity, hepatocytes, gate space (A), hepatic artery (B), and gate vein (C).

FIG. 1b shows a liver of the animal from the control group, evidencing tissue integrity, hepatocytes, and biliary duct (D).

FIG. 1c shows a liver of the animal from the experimental group (GROUP 1—single dose of 50 mg/kg), evidencing tissue integrity, hepatocytes, hepatic artery (B), and biliary duct (D).

FIG. 1d shows a liver of the animal from the experimental group (GROUP 1—single dose of 50 mg/kg), evidencing tissue integrity, hepatocytes, and vesicles of fat(E).

FIG. 1e shows a liver of the animal from the experimental group (GROUP 2—single dose of 300 mg/kg), evidencing hepatocytes and sinusoidal congestion (F).

FIG. 1f shows a liver of the animal from the experimental group (GROUP 2—single dose of 300 mg/kg), evidencing hepatocytes and sinusoidal congestion (F).

FIG. 1g shows a liver of the animal from the experimental group (GROUP 3—single dose of 2000 mg/kg), evidencing tissue integrity, hepatocytes, hepatic artery (B), and biliary duct (D).

FIG. 1h shows a liver of the animal from the experimental group (GROUP 3—single dose of 2000 mg/kg), evidencing tissue integrity, hepatocytes, hepatic artery (B), gate vein (C), and biliary duct (D).

In the administration tests of acyclovir along with pseudoboehmite in Wistar rats, the analysis of the blood of the rats, which was carried out by using a high performance liquid chromatography, showed that, in fact, there occurred the absorption of the acyclovir in the systemic circulation in those animals which received pseudoboehmite along with acyclovir.

The acyclovir administered along with pseudoboehmite was absorbed by the gastrointestinal tract. The acyclovir which was present in the plasma of the rats allowed to confirm that it is present in the systemic circulation of said animals even after the desorption of pseudoboehmite. The results showed that pseudoboehmite has a low short-term repeated-dose toxicity, and that it can be categorized as non-toxic. The plasma of the rats that received pseudoboehmite was analyzed by using atomic absorption spectrophotometry; the absence of aluminum in the plasma samples emphasizes the absence of absorption of aluminum to the systemic circulation.

Consequently, the results of the toxicity tests show that the pseudoboehmite has a low toxicity when administered at short-term, repeated-dose and therefore falls in the nontoxic category.

The tests of acyclovir administration in the presence of pseudoboehmite showed that the acyclovir was absorbed into the systemic circulation of the rats. Further, acyclovir presented in the plasma of the rats allowed to confirm that it is present in the systemic circulation of the animals even after the desorption of the pseudoboehmite.

1. Composition of a ceramic matrix with a controlled release drug, characterized in that the ceramic matrix is formed by pseudoboehmite nanoparticles, presenting a pharmaceutically acceptable degree of purity, a specific area of 250-300 mg²/g and defining 50% to 60% of the total composition weight, the drug comprising acyclovir to be controllably released in a human or animal organism and defining at least a portion of the remaining weight of the composition.

2. Composition, as set forth in claim 1, characterized in that it further comprises a pharmaceutically acceptable filler, a flow adjusting element and a lubricant agent.

3. Composition, as set forth in claim 2, characterized in that the pharmaceutically acceptable filler is defined by starch, which is present in an amount ranging from 20% to 30% in relation to the total weight of the composition.

4. Composition, as set forth in claim 2, characterized in that the flow adjusting element is defined by silicon dioxide, which is present in an amount ranging from 1.5% to 2% in relation to the total weight of the composition.

5. Composition, as set forth in claim 2, characterized in that the lubricant agent is defined by magnesium stearate, which is present in an amount ranging from 1.5% to 2% in relation to the total weight of the composition.

6. Tablet comprising a ceramic matrix carrying a controlled release drug, characterized in that the ceramic matrix is formed by pseudoboehmite nanoparticles, presenting a pharmaceutically acceptable degree of purity, a specific area of 250-300 mg²/g and defining 50% to 60% of the total tablet weight, the drug comprising acyclovir to be controllably released in a human or animal organism and defining at least a portion of a remaining weight of the tablet.

7. Tablet, as set forth in claim 6, characterized in that it further comprises pharmaceutically acceptable filler, a flow adjusting element and a lubricant agent.

8. Tablet, as set forth in claim 7, characterized in that the pharmaceutically acceptable filler is defined by starch, which is present in an amount ranging from 20% to 30% in relation to the total weight of the tablet.

9. Tablet, as set forth in claim 7, characterized in that the flow adjusting element is defined by silicon dioxide, which is present in an amount ranging from 1.5% to 2% in relation to the total weight of the tablet.

10. Tablet, as set forth in claim 7, characterized in that the lubricant agent is defined by magnesium stearate, which is present in an amount ranging from 1.5% to 2% in relation to the total weight of the tablet.

11. A method for obtaining a composition as defined in claim 1 and characterized in that it comprises, in a first phase, the production of a ceramic matrix of pseudoboehmite nanoparticles through the steps of:
mixing an aqueous aluminium nitrate or an aqueous aluminium chloride solution (14% m) with a poly(vinyl alcohol) solution (8% m in water), forming a precursor solution;
dripping the precursor solution in an ammonium hydroxide solution (28% m), forming a gel;
ageing the gel, filtering and drying it by about 70° C. for approximately 24 hours to obtain a ceramic matrix of pseudoboehmite presenting a specific area of 250-300 m²/gram; and, in a second phase, the step of
mixing the ceramic matrix of pseudoboehmite, in an amount from 50% to 60% of the total composition weight, with a controlled release drug comprising acyclovir to be controllably released in a human or animal organism and defining at least a portion of the remaining weight of the composition.

12. A method for producing a tablet as defined in claim 6 and characterized in that it comprises, in a first phase, the production of a ceramic matrix of pseudoboehmite nanoparticles, through the steps of:
mixing an aqueous aluminium nitrate solution or aqueous aluminium chloride solution (14% m) with a poly(vinyl alcohol) solution (8% m in water), forming a precursor solution;
dripping the precursor solution in an ammonium hydroxide solution (28% m), forming a gel;
ageing the gel, filtering and drying it at about 70° C. for approximately 24 hours to obtain a ceramic matrix of pseudoboehmite presenting a specific area of 250-300 m²/gram; and in a second phase, the steps of
mixing the ceramic matrix of pseudoboehmite, in an amount from 50% to 60% of the total tablet weight, with a controlled release drug comprising acyclovir to be controllably released in a human or animal organism and defining at least part of the remaining weight of the tablet; and
submitting the mixture ceramic matrix/acyclovir to a conformation under a pressure sufficient to form the tablet.

13. The method, as set forth in claim 12, characterized in that the step of mixing the ceramic matrix to the acyclovir comprises: mixing the ceramic matrix of pseudoboehmite with the drug and with the pharmaceutically acceptable filler and the flow adjusting element, during at least 15 minutes; adding the lubricant agent; and mixing the ceramic matrix and the acyclovir for at least 5 minutes before submitting said mixture to the step of press-formation.
